(19) United States
(12) Patent Application Publication
(10) Pub. No.: US 2012/0063564 A1
(43) Pub. Date: Mar. 15, 2012

(54) Method For 2D/3D Registration

(75) Inventor: Klaus Klingenbeck, Aufsess (DE)

(73) Assignee: SIEMENS AKTIENGESELLSCHAFT, Munich (DE)

(21) Appl. No.: 13/227,556

(22) Filed: Sep. 8, 2011

(30) Foreign Application Priority Data
Sep. 13, 2010 (DE) 10 2010 040 634.1

(51) Int. Cl. A61B 6/03; H05G 1/64

(52) U.S. Cl. 378/4; 378/98.12

(57) ABSTRACT

A method for overlaying a 3D volume image of a CT recording system and a 2D projection image of an angio recording system of an X-ray diagnostic facility including a rotatable gantry. The method includes providing a 3D volume image of an examination object reconstructed from a data record of the CT recording system and recording a 2D projection image of the examination object using the angio recording system. The method also includes determining a projection direction of the recorded 2D projection image in relation to the 3D volume image, based on the geometric arrangement between the recording systems. The method still further includes simulating a 2D projection image from the 3D volume image for the determined projection direction and matching the recorded 2D projection image and the simulated 2D projection image. The method includes overlaying the recorded 2D projection image and the 3D volume image, based on the matching.

### PRIORITY STATEMENT

The present application hereby claims priority under 35 U.S.C. §119 on German patent application number DE 10 2010 040 634.1 filed Sep. 13, 2010, the entire contents of which are hereby incorporated herein by reference.

### FIELD

At least one embodiment of the invention generally relates to a method for registering a 3D volume image of a CT recording system with at least one 2D projection image of an angio recording system of an X-ray diagnostic facility.

### BACKGROUND

X-ray diagnostic systems are a standard procedure in medical imaging and are used for example for interventional therapy. Angiography systems, generally C-arm X-ray systems, are used for example to monitor the treatment of vascular and cardiac diseases and for the minimally invasive treatment of tumors. With their flat panel X-ray detectors with pixel element matrix arrangements they provide a very high spatial resolution (pixel size generally around 150 μm) and can be used both for 2D and 3D imaging (Siemens DynaCT). However for low contrast resolution and recording speed conventional computed tomography systems still have the edge in 3D imaging but they have disadvantages in respect of resolution and recording field in 2D imaging.

In order to be able to utilize the advantages of both systems, an X-ray diagnostic apparatus is known for example from DE 198 02 405 B4, in which two recording systems are disposed on a rotatable gantry—a CT recording system with a row type X-ray detector and an angio recording system with a flat surface type X-ray detector. The CT recording system can be used to implement the known CT modes, e.g. the recording of sequential slices with what is known as the stop and shoot advancing of a patient table or spiral imaging with continuous advancing of the patient table and continuous gantry rotation. With the angio recording system it is possible to implement two known modes—2D fluoroscopy imaging with a stationary gantry and 3D rotational imaging (e.g. DynaCT) with a continuously or sequentially rotating gantry.

### SUMMARY

At least one embodiment of the present invention provides a particularly exact method for registering 3D volume images and 2D projection images with an X-ray diagnostic apparatus having a CT recording system and an angio recording system, it being possible to use the method to monitor an interventional procedure on an examination object in a simple manner.

According to at least one embodiment of the invention, a method is disclosed for registering a 3D volume image of a CT recording system with at least one 2D projection image of an angio recording system of an X-ray diagnostic facility. Advantageous embodiments of the invention are set out respectively in the subclaims.

At least one embodiment of the inventive method is directed to a method for registering a 3D volume image of a CT recording system with at least one 2D projection image of an angio recording system of an X-ray diagnostic facility having a rotatable gantry, said recording systems being disposed together in the gantry, the CT recording system featuring a first X-ray source and a computed tomography X-ray detector having a row of individual detectors and being disposed opposite the first X-ray source and the angio recording system featuring a second X-ray source, which is disposed offset in relation to the first X-ray source, and a flat surface type X-ray detector with a pixel element matrix arrangement disposed opposite the second X-ray source, the method comprises:
Providing a 3D volume image of an examination object reconstructed from a data record of the CT recording system,
Recording a 2D projection image of the examination object using the angio recording system,
Determining the projection direction of the recorded 2D projection image in relation to the 3D volume image taking account of the geometric arrangement between CT recording system and angio recording system,
Simulating a 2D projection image (in particular DRR=Digitally Reconstructed Radiograph) from the 3D volume image for the determined projection direction,
Matching the recorded 2D projection image and the simulated 2D projection image, and
Overlaying the recorded 2D projection image and the 3D volume image taking account of the match.

### BRIEF DESCRIPTION OF THE DRAWINGS

The invention and further advantageous embodiments according to the features of the subclaims are described in more detail below based on schematically illustrated example embodiments in the drawing, without the invention being restricted to said exemplary embodiments. In the drawings:

FIG. 1 shows a known X-ray diagnostic apparatus having two recording systems and

FIG. 2 a sequence of an embodiment of the inventive method.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

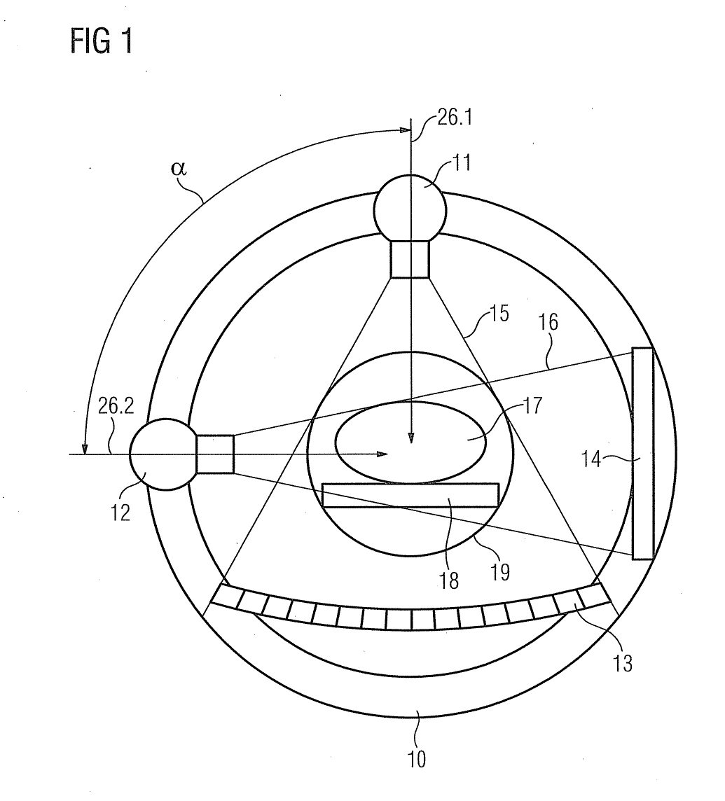

A known X-ray diagnostic apparatus shown in FIG. 1 features in a gantry 10 a computed tomography recording system having a first X-ray source 11 and a CT X-ray detector 13 and an angiography recording system having a second X-ray source 12 and a second flat image X-ray detector 14. In the CT recording system the first X-ray source 11 transmits a fan beam 19 in the first projection direction 26.1 and the CT X-ray detector 13 is curved and made up of a row of individual detectors (e.g. 512). To scan an examination object 17 positioned on a patient table 18, the CT recording system is rotated by way of the gantry 10 through 360° about the examination object 17; the recorded data record can be reconstructed to provide a 3D volume image.

The angio recording system features a second X-ray source 12 and a flat image X-ray detector 14 and the second X-ray source transmits a cone-shaped X-ray beam 16 in the second projection direction 26.2 onto the flat image X-ray detector 14. Between the first projection direction 26.1 and the second projection direction 26.2 is an offset angle α, which can be used to describe the offset between the CT recording system and the angio recording system. The angio recording system can be used with the gantry stationary to record 2D projection images and with the gantry rotating to record a projection image data record that can be reconstructed to provide a 3D image. It is possible to operate the CT recording system and the angio recording system simultaneously or alternately, as described for example in DE 198 02 405 B4, the entire contents of which are hereby incorporated herein by reference.

To actuate the X-ray diagnostic facility a system controller is provided for example, which actuates both the CT recording system and the angio recording system. Such a system controller can be formed by a control PC.

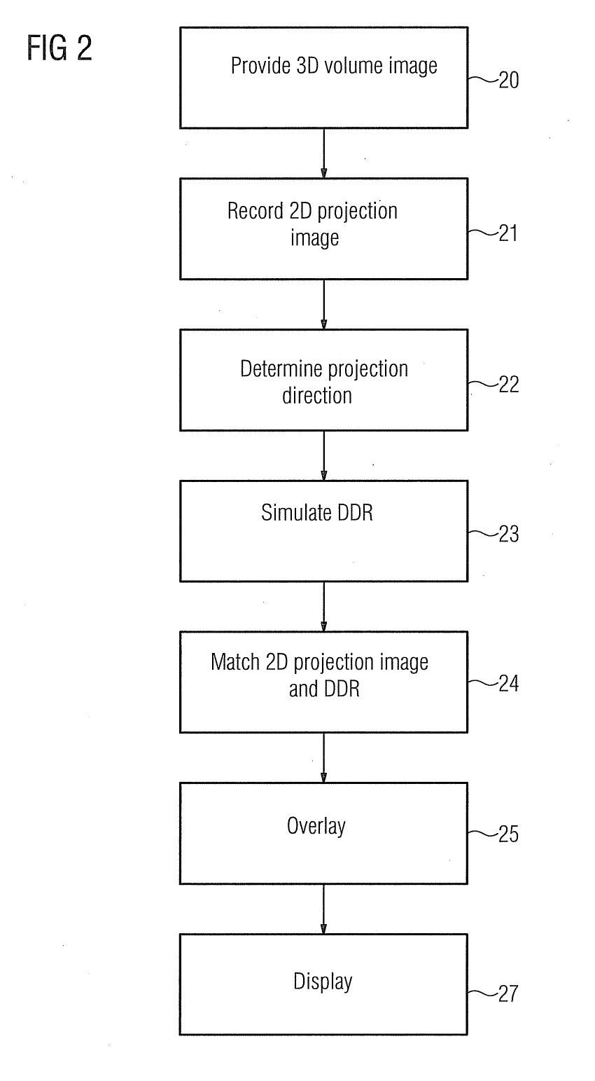

FIG. 2 shows a sequence of an embodiment of an inventive method for registering a 3D volume image of the CT recording system with at least one 2D projection image of the angio recording system of the X-ray diagnostic facility shown in FIG. 1. An embodiment of the inventive method can be actuated for example automatically by the system controller of the X-ray diagnostic facility. For reconstructions and simulations or further calculations it is possible to use a computation unit actuated by the system controller. In a first step 20, which does not necessarily have to take place before the following step, a 3D volume image of an examination object reconstructed from a data record of the CT recording system is provided. Such provision can entail for example either retrieving a recording recorded at a previous time from a storage unit of the X-ray diagnostic facility or a new recording of a data record using the CT recording system and corresponding reconstruction of the data record to provide a 3D volume image.

In a second step 21 at least one 2D projection image of the examination object is recorded using the angio recording system. In a third step 22 the projection direction of the recorded 2D projection image is determined in relation to the 3D volume image taking account of the geometric arrangement between the CT recording system and the angio recording system. Since the geometric arrangement between the CT recording system and the angio recording system on the gantry is fixed and known, given that the offset angle α for example is known, it is possible in a simple manner to derive the projection direction of the angio recording system relative to the CT recording system and in this manner to determine the projection direction in the 3D volume image.

In a fourth step 23 a 2D projection image is then simulated for the determined projection direction of the angio recording system, e.g. by way of a computation unit, from the 3D volume image or the data record, from which the 3D volume image was reconstructed. Such 2D projection images simulated from CT data records are known and are also referred to as DRR (Digitally Reconstructed Radiograph). In a fifth step 24 the 2D projection image recorded using the angio recording unit and the 2D projection image simulated from the 3D data record for the same projection direction are matched, by for example taking account of zoom and enlargement factors or further image and recording information. In a sixth step 25 the recorded 2D projection image is overlaid with the 3D volume image using the simulated 2D projection image so that registration is completed. Optionally in a seventh step 27 the overlaid images can be displayed for example on a display unit of the X-ray diagnostic facility.

An embodiment of the inventive registration can be performed particularly advantageously in conjunction with an interventional procedure or catheter navigation before or during the intervention. The 3D volume image here can serve as a road map and after registration 2D projection images recorded online can be shown continuously in the 3D volume image in a simple manner, so that the progress of the intervention is clearly visible and easy to monitor. Generally it is very advantageous to perform such a registration at the start of or directly before an intervention or navigation.

The 3D-volume image determined using the angio recording system can be used as a 3D road map for navigating devices (devices being needles or catheters for example). The insertion and advancing of the devices can be shown and followed by way of fluoroscopy using the angio recording system. The advantage of an embodiment of the inventive registration is that the 3D volume image of the CT recording system is projected with geometric precision onto the 2D projection image (fluoroscopy image) of the angio recording system, allowing the progress of the device to be displayed and tracked in real time in the 3D volume image. This simple and robust method can be applied, since in the present X-ray diagnostic facility the CT recording system and the angio recording system are fixed permanently in relation to one another on the gantry. The CT volume image is a geometrically exact and precise digital representation of the examination object. Since the position of the angio recording system in space relative to the CT recording system is known, it is possible first to determine the projection direction that brings about the projection of the 3D volume onto the 2D projection image exactly.

The actual projection can be performed using known methods, e.g. Digitally Reconstructed Radiographs (DRR). The DDR is then matched to the geometry of the angio recording system. Since the system controller knows the geometry of the angio recording system, e.g. the focal point to detector distance, the geometric enlargement of the angio recording system is known and can be taken into account during or after calculation of the DRR. This allows exact overlaying of the projected 3D volume with the fluoroscopy to be achieved. Should significant patient movement occur during the intervention, the recording of a 3D volume image using the CT recording system can be updated generally or locally and an embodiment of the inventive registration method can be repeated.

An embodiment of the invention can be summarized briefly as follows: to improve 2D/3D registration a method is provided for registering a 3D volume image of a CT recording system with at least one 2D projection image of an angio recording system of an X-ray diagnostic facility having a rotatable gantry, said recording systems being disposed together in the gantry, the CT recording system featuring a first X-ray source and a computed tomography X-ray detector having a row of individual detectors and being disposed opposite the first X-ray source and the angio recording system featuring a second X-ray source, which is disposed offset in relation to the first X-ray source, and a flat surface type X-ray detector with a pixel element matrix arrangement disposed opposite the second X-ray source, the method comprising:
Providing a 3D volume image of an examination object reconstructed from a data record of the CT recording system,
Recording a 2D projection image of the examination object using the angio recording system,
Determining the projection direction of the recorded 2D projection image in relation to the 3D volume image taking account of the geometric arrangement between CT recording system and angio recording system,
Simulating a 2D projection image (Digitally Reconstructed Radiograph) from the 3D volume image for the determined projection direction,
Matching the recorded 2D projection image and the simulated 2D projection image, and
Overlaying the recorded 2D projection image and the 3D volume image taking account of the match.

At least one embodiment of the inventive method allows a particularly exact, simple and robust 2D/3D registration to be performed, with the fixed geometric arrangement of the two recording systems in relation to one another being utilized to accelerate the registration. The particularly precise and error-free assignment of 3D volume image and 2D projection images results in a particularly low error rate during diagnosis and therefore to a high level of reliability for an examined patient.

According to one embodiment of the invention, the 3D volume image and the overlaid 2D projection image are displayed on a display unit. The X-ray diagnostic apparatus and the two recording systems are advantageously actuated by a system controller. The system controller can also perform the individual steps automatically so that a user can concentrate totally on other actions, for example the performance of a navigation or an intervention on the patient.

According to a further embodiment of the invention, the enlargement used when recording the 2D projection image is taken into account when matching the recorded 2D projection image and the simulated 2D projection image. This enlargement can be determined for example from the geometric configuration, for example the distance between the second X-ray source and the flat image X-ray detector, of the angio recording system.

Further 2D projection images of the examination object are advantageously recorded using the angio recording system and overlaid with the 3D volume image. For these sequential images overlaying is considerably simplified by registration, as a simulated 2D projection image (Digitally Reconstructed Radiograph) is already available, so that overlaying of the 3D volume image and the recorded 2D projection image can be performed quickly and simply. In the case of a new 3D volume image a new 2D projection image is simulated and new matching and overlaying then follow.

According to a further embodiment of the invention the inventive method is performed before or during an interventional procedure or catheter navigation on the examination object to monitor said intervention or navigation. Further 2D projection images are then recorded for online monitoring, these then being overlaid with the 3D volume image so that it is possible to monitor the progress of the interventional procedure or catheter.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A method for overlaying a 3D volume image of a CT recording system and at least one 2D projection image of an angio recording system of an X-ray diagnostic facility including a rotatable gantry, the CT and angio recording systems being disposed together in the gantry, the CT recording system including a first X-ray source and a computed tomography X-ray detector including a row of individual detectors and being disposed opposite the first X-ray source, and the angio recording system including a second X-ray source, disposed offset in relation to the first X-ray source, and a flat surface type X-ray detector with a pixel element matrix arrangement disposed opposite the second X-ray source, the method comprising:
providing a 3D volume image of an examination object reconstructed from a data record of the CT recording system;
recording a 2D projection image of the examination object using the angio recording system;
determining a projection direction of the recorded 2D projection image in relation to the 3D volume image, taking account of a geometric arrangement between CT recording system and angio recording system;
simulating a 2D projection image from the 3D volume image for the determined projection direction;
matching the recorded 2D projection image and the simulated 2D projection image; and
overlaying the recorded 2D projection image and the 3D volume image, taking account of the matching.

2. The method as claimed in claim 1, wherein the 3D volume image and the 2D projection image are displayed on a display unit.

3. The method as claimed in claim 1, wherein the X-ray diagnostic facility and the CT and angio recording systems are actuated by a system controller.

4. The method as claimed in claim 1, wherein the method is performed automatically.

5. The method as claimed in claim 1, wherein an enlargement used, when recording the 2D projection image, is taken into account during the matching of the recorded 2D projection image and the simulated 2D projection image.

6. The method as claimed in claim 1, wherein further 2D projection images of the examination object are recorded using the angio recording system and are overlaid with the 3D volume image.

7. The method as claimed in claim 1, wherein the method is performed during an interventional procedure on the examination object, to monitor the intervention.

8. The method as claimed in claim 2, wherein the X-ray diagnostic facility and the CT and angio recording systems are actuated by a system controller.

9. A tangible computer readable medium including program segments for, when executed on a computer device, causing the computer device to implement the method of claim 1.

10. A method, comprising:
recording a 2D projection image of a 3D volume image an examination object, reconstructed from a data record of a CT recording system, using an angio recording system, the CT and angio recording systems being disposed together in a gantry;
determining a projection direction of the recorded 2D projection image in relation to the 3D volume image, taking account of a geometric arrangement between CT recording system and angio recording system;
simulating a 2D projection image from the 3D volume image for the determined projection direction;
matching the recorded 2D projection image and the simulated 2D projection image; and
overlaying the recorded 2D projection image and the 3D volume image, taking account of the matching.

11. The method as claimed in claim 10, further comprising:
displaying the 3D volume image and the 2D projection image on a display unit.

12. The method as claimed in claim 10, wherein the CT and angio recording systems are actuated by a system controller.

13. The method as claimed in claim 10, wherein the method is performed automatically.

14. The method as claimed in claim 10, wherein an enlargement used, when recording the 2D projection image, is taken into account during the matching of the recorded 2D projection image and the simulated 2D projection image.

15. The method as claimed in claim 10, wherein further 2D projection images of the examination object are recorded using the angio recording system and are overlaid with the 3D volume image.

16. The method as claimed in claim 10, wherein the method is performed during an interventional procedure on the examination object, to monitor the intervention.

17. The method as claimed in claim 11, wherein the CT and angio recording systems are actuated by a system controller.

18. A tangible computer readable medium including program segments for, when executed on a computer device, causing the computer device to implement the method of claim 10.
